(19) United States
(12) Patent Application Publication
(10) Pub. No.: US 2024/0027345 A1
(43) Pub. Date: Jan. 25, 2024

(54) DETECTION OF VOLATILE ORGANIC COMPOUNDS IN AIR

(71) Applicant: Koniku Inc., San Rafael, CA (US)

(72) Inventors: Oshiorenoya E. AGABI, San Rafael, CA (US); Jack Schenkman, San Rafael, CA (US)

(21) Appl. No.: 17/872,278

(22) Filed: Jul. 25, 2022

(51) Int. Cl. G01N 21/64; G01N 33/00; C12M 3/06; C12M 1/00; C12M 1/32; C12M 1/12

(52) U.S. Cl. CPC G01N 21/6428; G01N 33/0047; C12M 23/16; C12M 29/10; C12M 23/12; C12M 25/02; G01N 2021/6439

(57) ABSTRACT

A biochip includes a perfusion layer between a media layer and a first membrane. The perfusion layer has a perfusion channel to provide a liquid to cells in a well layer, with the cells attached to a second membrane below the well layer. A stimulation layer below the second membrane includes a stimulation air channel having a stimulation channel inlet and a stimulation channel outlet. The biochip may be used to detect volatile organic compounds in air.

The field of the invention is detection of volatile organic compounds (VOCs).

### BACKGROUND OF THE INVENTION

Volatile organic compounds (VOCs) are natural or manmade compounds which readily diffuse into air, due to their volatile characteristics. Many VOCs are toxic to humans and to the environment with extended exposure. VOCs are also associated with explosives. Thus, detecting VOCs is important to human safety and security, and for better preserving the environment. Although techniques have been proposed and used for detecting VOCs, they have been met with only varying degrees of success. Accordingly, improved systems and methods for detecting VOCs in air are needed.

### OVERVIEW

A system for detecting VOC's uses living genetically modified biological cells. In humans, the sense of smell is generally achieved by a type of neuron located in the nasal epithelium, which express olfactory or odorant receptors (OR) on their surfaces. Each odorant neuron usually expresses only one OR gene among the hundreds present in the organism's genome. When an odorant molecule, or VOC, from inhaled air binds to a matching receptor, the event triggers a chain of reactions that result in electrical signals. These signals, or spikes, propagate into the brain and are further processed to give rise to a complex sense of smell.

A cell may be modified to express a receptor. The receptor may be an odorant or a wild-type receptor. The receptor may be a modified receptor, such as a receptor genetically modified to enhance a binding specificity to a particular compound or to alter the receptor from a broadly tuned receptor to a narrowly tuned receptor or vice versa. The cell may be modified to express only one unique receptor, or more than one unique receptor, e.g., two, three or more receptors. A receptor may be a human receptor, a mouse receptor, a canine receptor, an insect receptor, or other species type of odorant receptor.

OR activation eventually results in an increase in cytosolic calcium concentration, which can be measured using a calcium sensitive fluorescent reporter. These may include FIP-CBSM, Pericams, GCaMPs TN-L15, TNhumTnC, TN-XL, TN-XXL, Twitch's, RCaMP1, jRGECO1a, or any other suitable genetically encoded calcium indicator. The binding of an odorant molecule to its receptor induces an increase in the fluorescence emitted by the cells. An optical detector can therefore be used to measure cellular response in a contactless manner. The present system and methods can detect VOC's using an optical detector that detects fluorescence.

A biochip used in the present system has one or more wells containing genetically modified living cells expressing an odorant receptor capable of binding to a volatile organic compound, and a fluorescent reporter that fluoresces in response to binding of the volatile organic compound to the odorant receptor. An air flow channel is separated from each well by a membrane. Living cells are bound to a first side of the membrane, and a surface of the airflow channel is formed by a second side of the membrane. At least a portion of the biochip may be transparent.

Other objects, features and advantages will become apparent from the following detailed description and drawings, which are provided as examples for explanation, and are not intended to be limits on the scope of the invention.

### BRIEF DESCRIPTION OF THE DRAWINGS

In the drawings, the same element number indicates the same element in each of the views.

FIG. 1 is a schematic diagram of a VOC detection system.

FIG. 2 is a schematic diagram of the optical system of the VOC detection system of FIG. 1.

FIG. 3A is an exploded perspective view of a microfluidic biochip.

FIG. 3B shows a modification of the microfluidic biochip of FIG. 3A.

FIG. 4 is an exploded perspective view of another microfluidic biochip.

FIG. 5 is an exploded view of yet another microfluidic biochip.

FIG. 6 is a perspective view of the media layer shown in FIG. 3A.

FIG. 7 is a perspective view of the perfusion layer shown in FIGS. 3 and 5.

FIG. 8 is a perspective view of the upper well layer shown in FIGS. 3 and 4.

FIG. 9 is a perspective view of the lower well layer shown in FIGS. 3 and 4.

FIG. 10 is a perspective view of the stimulation layer shown in FIGS. 3 and 4.

FIG. 11 is a perspective view of the upper stimulation layer shown in FIG. 5.

FIG. 12 is a perspective view of the well layer shown in FIG. 5.

FIG. 13 is a perspective view of the lower membrane shown in FIG. 5.

FIG. 14 is a perspective view of the lower stimulation layer shown in FIG. 5.

FIG. 15 is a perspective view of an alternative perfusion layer having a single port.

FIG. 16 is an exploded perspective view of another microfluidic biochip.

### DETAILED DESCRIPTION

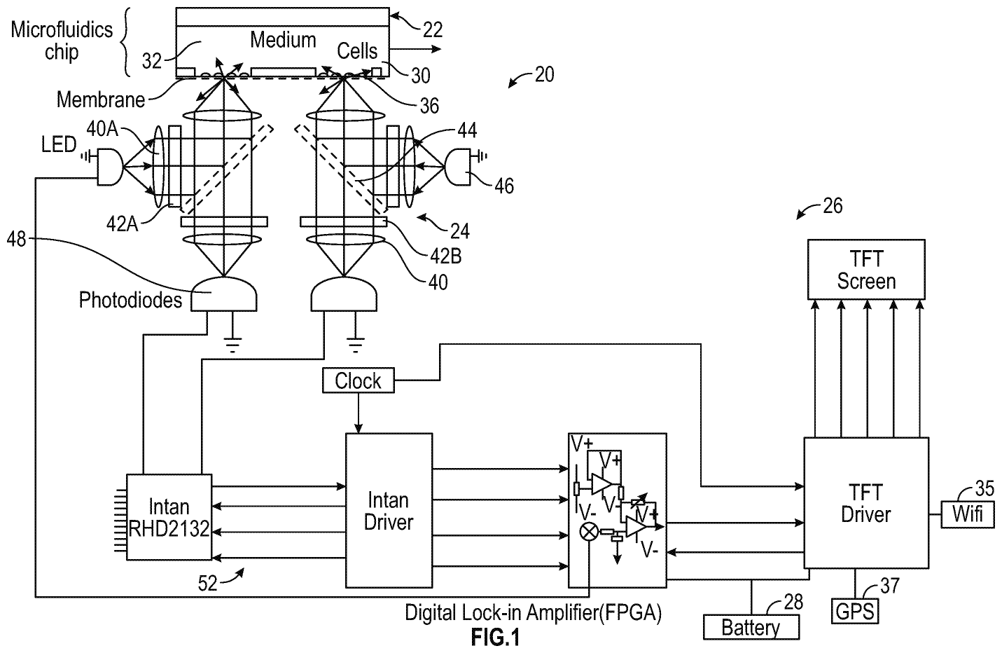

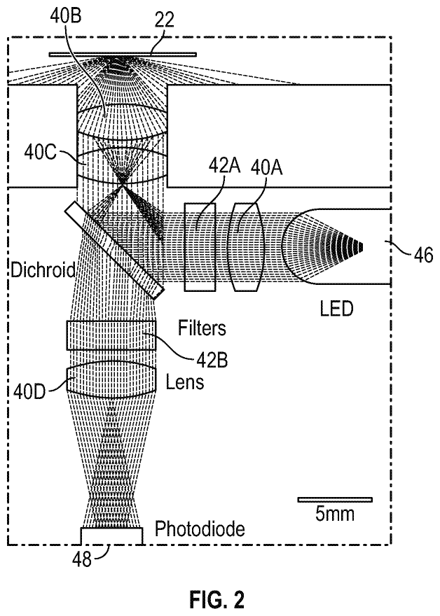

Referring to FIGS. 1 and 2, in a basic form, a VOC detection system 20 includes a cell carrier or substrate, such as a microfluidic biochip 22, an optical system 24 and an electronic system 26. The microfluidic biochip 22 contains cells 30 in a liquid medium 32. The cells bind to the membrane 36, allowing the cells to more effectively interact with airborne odorants such as VOC's, which diffuse through the membrane. Each channel or optical pathway of the optical system 24 includes one or more: light emitter, such as a blue LED 46, lenses 40A, 40B 40C and 40D, optical filters 42A and 42B, dichroic mirror 44, and a photodetector such as a photodiode 48.

FIG. 1 shows an embodiment having two optical pathways each having the above-listed elements, although the system may be designed with a single optical pathway or multiple optical pathways, depending on the intended application. The electronic system 26 in FIG. 1 is electrically connected to the blue LEDs 46 and to the photodiodes 48 and may include a digital lock-in amplifier 51 in the form of a field programmable gate array (FPGA). The electronic system 26 has an output device, such as a thin film transistor (TFT) display. Alternatively, the output or reporting from the detection system 20 may be provided via a WIFI 35, cellular, RF or wired connection. The electronic system 26 may include a GPS unit 37 for detecting and reporting the location of the detection system 20. The electronic system 26 may also include control software or circuitry, and memory for recording detection events and other data. The detection system 20 may be powered by a battery 28, to allow flexibility in placement and use.

The system shown in FIGS. 1 and 2 can operate with microfluidic biochips of varying designs, for example with the microfluidic biochip described in U.S. patent application Ser. No. 17/571,363, incorporated herein by reference.

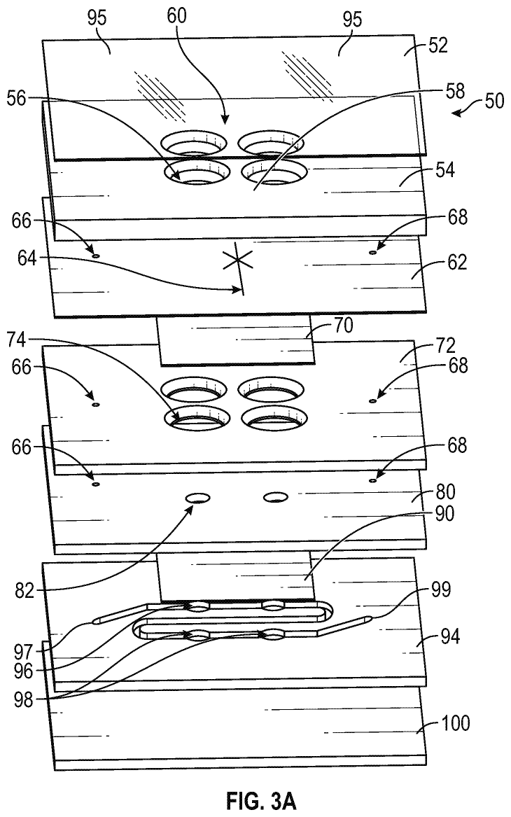

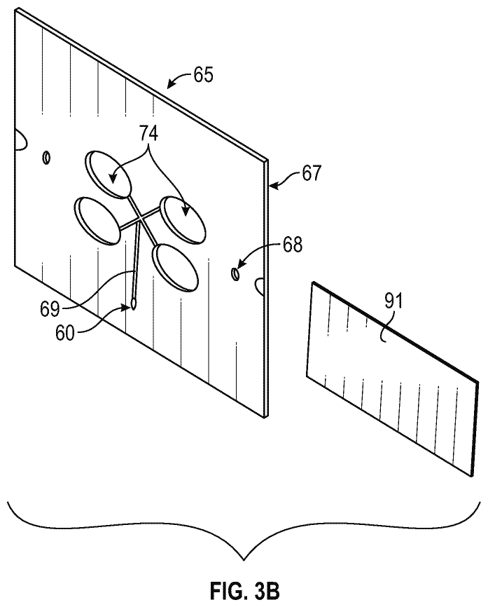

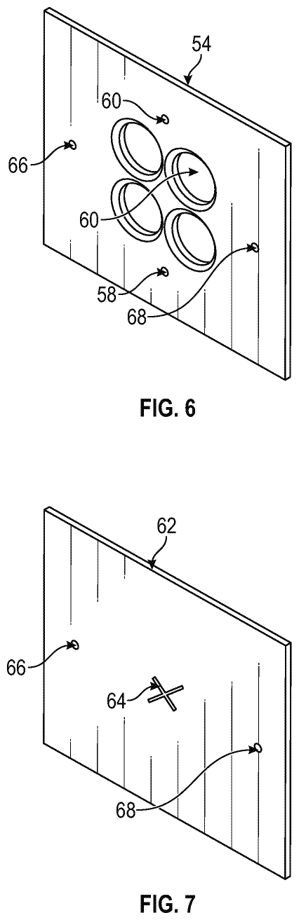

FIG. 3A shows an alternative biochip 50 which may include multiple layers. Referring to FIGS. 3A and 6, in the biochip 50 a cover 52 is attached on top of a media layer 54. The cover 52 may a silicon or plastic film, or a metal foil. The bottom surface of the cover 50 may be reflective, to increase light detectable by the detection system 20. The cover 52 seals the biochip 50, excluding contaminants and preventing the cell media from evaporating. Referring also to FIGS. 7 and 8, a perfusion layer or perfusion layer 62 and an upper or first membrane 70 are between the media layer 54 and an upper well layer 72 having wells or through holes 74. The upper membrane 70 provides a permeable barrier between the media of the media layer 54 and the media contained below it in the cell wells 74. The media layer 54 has media wells 56 holding media. The media wells are aligned over the cell wells 74. In some embodiments, the cover 52 may be transparent, with the detection system 20 projecting light into, and/or detecting cell fluorescence from above rather than from below as shown in FIGS. 1 and 2.

Perfusion inlet hole(s) 58 and perfusion outlet hole(s) 60 extend from the cover 52 through the media layer 54 and lead into a first or perfusion inlet channel and a second or perfusion outlet channel 64 in the perfusion layer 62. The perfusion layer 62 has perfusion channels 64 connecting to the perfusion inlet holes 58 and perfusion outlet holes 60 and into the cell wells 74. Before the biochip is used, the perfusion inlet hole(s) 58 and the perfusion outlet hole(s) 60 may be closed off or sealed by the cover 52. The system 20 has perfusion media inlet and outlet tubes which move to pierce or puncture into or through the cover 52 and connect with the perfusion inlet and outlet channels, after the biochip is installed in the system 20, as described in U.S. patent application Ser. No. 17/571,363.

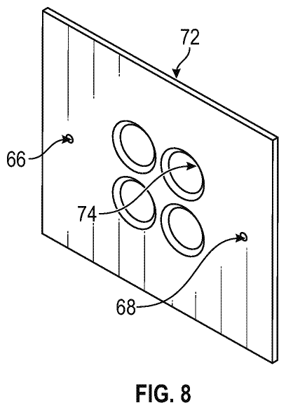

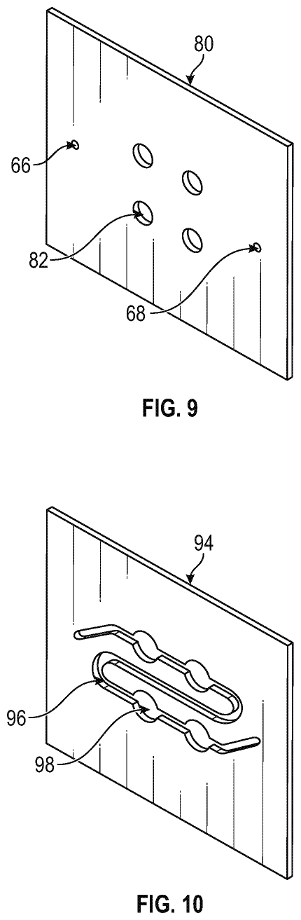

Referring to FIGS. 3A, 9 and 10, a lower well layer 80 and a lower or second membrane 90 are between the upper well layer 72 and a stimulation layer 94 having an air or stimulation channel 96. The stimulation channel 96 has a stimulation channel inlet 97 at a first end and a stimulation channel outlet 99 at a second end.

The stimulation channel 96 may have circular well regions 98 aligned under the cell wells 74. A gas or air inlet hole 66 and a gas or air outlet hole 68 extends through each of the layers above the stimulation layer 94, to provide air or gas through the stimulation channel 96 from gas fittings 95 on or inserted into the top surface or layer biochip 50 when used in a system. The well regions 98, if used, may be provided as holes having a diameter greater than the width of the stimulation channel 96. The well regions 98 are then aligned with well holes 82 in the lower well layer 80 and the cell wells 74.

The perfusion layer 62 may be a polyester film with holes, and slots for the perfusion channels 64. The well regions expose a larger surface area of the lower membrane 90, and the cells on it, to the gas in the stimulation channel 96.

The stimulation layer 94 is attached to a bottom layer 100 which closes off or seals off the stimulation channel 96 from below. The bottom layer 100 may have a rough surface, or have projections or obstacles projecting up into the stimulation channel 96 to promote turbulent flow of gas through the stimulation channel 96. The bottom layer 100 may have no openings or other features and consequently is not separately illustrated.

The lower well layer 80 may optionally be combined with the upper well layer 72, with both provided as a single layer or component. Similarly, the lower well layer 80 and the upper well layer 72 may be provided as a single layer or component. In some designs, the perfusion layer may be part of, or incorporated into, the media layer. The thickness of the upper well layer 72 may be minimized to e.g., 0.2 to 1 or 2 mm to better allow fresh media to more easily diffuse into the cell wells 74.

The perfusion inlet hole 58, the perfusion outlet hole 60, and the holes for the gas fittings 95, if used, or the gas inlet hole 66 and gas outlet hole 68 may be sealed by the cover 52 at the top of the biochip. These openings are accessed via the detection system puncturing or piercing through the cover 52. The membranes 70 and 90 are semipermeable in that they allow gases and liquids to pass through.

As shown in FIG. 3A, all of the layers may be thin flat rectangular slabs having the same length and width, except for the membranes 70 and 90 which may square, and just large enough to cover the cell wells 74. In some designs, all of the layers and the membranes may have the same size and shape, with the membranes 70 and 90 having the same length and width dimensions as the other layers, or with the membrane material within a surrounding frame of other material having the same length and width as the other layers.

FIG. 3B shows a modified design where a perfusion layer, a well layer and a membrane are combined into a single layer 65. The cell wells 74 are provided as holes through the sheet 67. The wells in the layer 65 are shown as oval, but may also be round. The layer 65 may be provided as a membrane 91 attached to a glass or plastic sheet 67. The sheet 67 may have adhesive top and bottom surfaces, to adhere to adjoining layers. A perfusion inlet channel 69 is cut or etched into the top surface of the sheet 67 and connects a perfusion inlet hole 58 with each of the cell wells 74. A perfusion outlet channel 71 is cut or etched into the bottom surface of the sheet 67 and connects each of the cell wells 74 to perfusion outlet hole 60. The membrane 91 is attached or adhered to the bottom surface of the sheet 67 and overlies the perfusion outlet channel 71. The flow direction of perfusion liquid through the layer 65 may be reversed so that incoming perfusion liquid enters each well 74 adjacent to the membrane 91, with perfusion liquid removed from the wells via the perfusion outlet channel 71 above the membrane 91, on which the cells are located. Flow of perfusion liquid may alternate between flowing in and flowing out through the same channel.

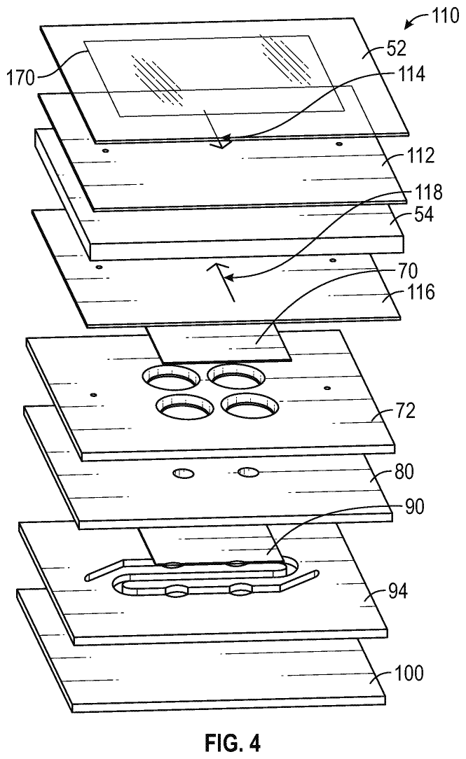

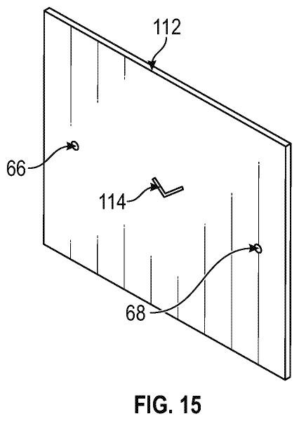

Referring to FIGS. 4 and 15, an alternative biochip 110 may be the same as the biochip 50 shown in FIG. 3A, except the biochip 110 has a first or upper perfusion layer 112 having a first perfusion channel 114 between the cover 52 and the media layer 54, and also a second or lower perfusion layer 116 having a second perfusion channel 118 between the media layer 54 and the upper membrane 70. Thus, the two perfusion layers 112 and 116 are on different vertical levels. Either of the two perfusion layers 112 and 116 may be a perfusion inflow layer, with the other being a perfusion outflow layer. In example of FIG. 4 all of the layers may be transparent. Alternatively, only the bottom 100 and the membrane 90 are transparent.

Referring still to FIG. 4, in any of the biochips described, a heating element 170 may be included to maintain the cells at a desired temperature. The heating element 170 may be an integral part of the biochip, with electrical contacts at the top or bottom of the biochip. Alternatively, a heating element 170 may be provided on the biochip reader or apparatus, which inserts the heating element 170 into a hole in the biochip. Multiple heating elements may be used to provide more uniform heating to all of the wells.

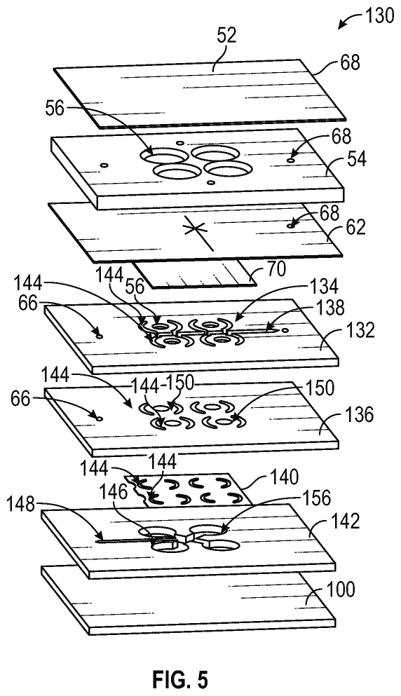

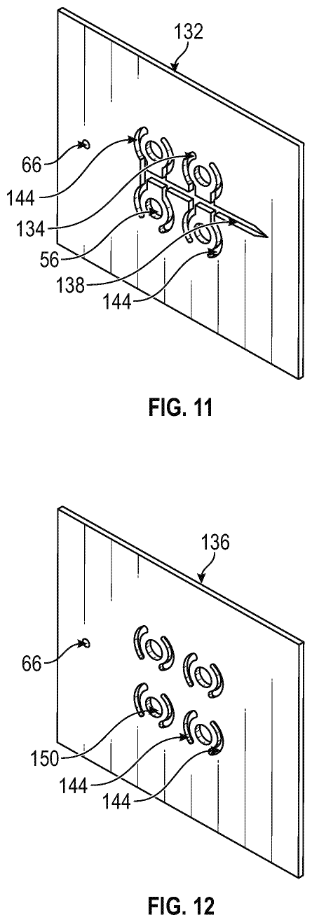

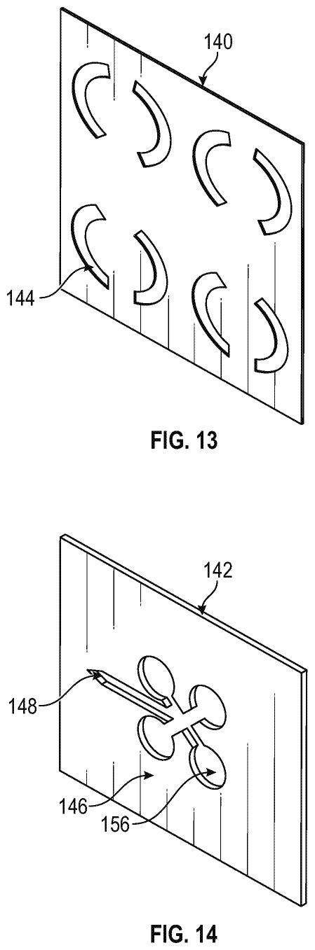

FIGS. 5 and 11-14 show another alternative biochip 130 which, like the biochip 50 shown in FIG. 3A, may have a cover 52 on a media layer 54, a single perfusion layer 62 and an upper membrane 70. In addition, the biochip 130 has an upper or first stimulation layer 132 and a lower or second stimulation layer 142. In this example, a single perfusion layer 62 is shown, although two perfusion layers may optionally be used. One or more opaque perfusion layers may also optionally be used. The upper stimulation layer 132 has an upper or first stimulation channel 134 including vertical pass through openings 144 all connected into an air outflow segment 138, as shown in FIG. 11. The vertical pass through openings 144 may be provided as crescent shaped openings or slots. The air outflow segment 138 connects into air outlet holes 68 in the layers above the upper stimulation layer 132. The well layer 136 below the upper stimulation layer 132 includes wells 150 and may be the same as the well layer 72, with the addition of vertical pass through openings 144.

Referring back to FIG. 5, a lower membrane 140 is between the lower stimulation layer 142 and the well layer 136. The lower stimulation layer 142 has a first or lower stimulation channel 146 including an air inlet segment 148 leading into well regions 156 aligned under the wells 150. The lower stimulation channel 146 connects with a second or upper stimulation channel 134 via the vertical pass through openings 144 in the lower membrane 140, the well layer 136 and the upper stimulation layer 132. The vertical pass through openings 144 in the upper stimulation layer 132 need not extend vertically entirely through the upper stimulation layer 132, as they are shown in FIG. 5, because they only need to connect the vertical pass through openings 144 in the well layer 136 to the air outflow segment 138. The air inlet segment 148 connects with the gas inlet holes 66 extending through the layers above the lower stimulation layer 142.

In the biochip 130 shown in FIG. 5, in use air or gas including or carrying a VOC sample is moved down to the air inlet segment 148 of the lower stimulation channel, through the gas inlet holes 66 in the layers above the lower stimulation layer 142. The air then flows laterally to the well regions 156 and then vertically up through the vertical pass through openings 144 and into the upper stimulation channel 134, through the air outlet segment, and then out of the biochip via the air outlet holes 68 and fitting 95, if used.

As a result, the air or gas, carrying or driving VOC samples, flows through the biochip 130 and impinges directly against the bottom side of the lower membrane 140, inducing movement of air molecules through the lower membrane 140. The air molecules contact the cells, which are attached to the upper surface of the lower membrane 140. After impinging perpendicularly against the lower membrane 140, the air then flows generally parallel to the membrane and radially outward to the vertical pass through openings 144, into the upper stimulation channel 134 and then out of the biochip 130. This design may provide improved contact between the VOC sample in the air flow and the cells in the wells, leading to better sensitivity or detection accuracy.

Although the examples above describe biochips having four wells, the biochips may of course have other numbers of wells. Any of features and elements described above relative to one embodiment may also of course be used any of the embodiments disclosed.

In each of the biochips described, the layers may be laser cut from PET plastic sheets (polyethylene terephthalate) or other materials, such as silicon, fused-silica, glass, any of a variety of polymers, e.g., polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), epoxy resins, metals (e.g., aluminum, stainless steel, copper, nickel, chromium, and titanium), or any combination of these materials.

The layers may be attached and sealed together via an adhesive, solvent welding, clamping or by using bio-compatible double sided tape and a hot press. The layers may optionally be made of glass and/or PDMS (silicon-based organic polymer) assembled using plasma bonding. The layers may be translucent or transparent.

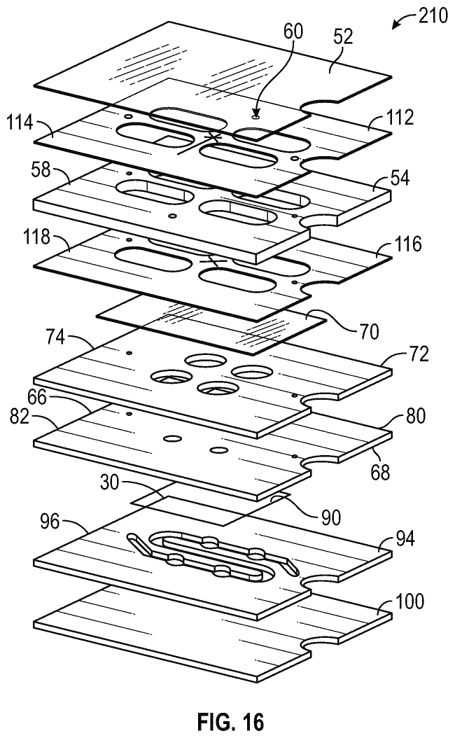

FIG. 16 shows an exploded view of another biochip 210 having elements in common with the biochip 110 in FIG. 4. In FIG. 16 the biochip 210 has the following layers attached to each other in the sequence shown, from the top to the bottom: a top or seal layer 52; an upper or first perfusion layer 112; a media layer 54; a lower or second perfusion layer 116; a top or first membrane 70; an upper well layer 72; a lower well layer 80; a lower or second membrane 90; a stimulation layer 94 and a bottom layer 100. Although the upper perfusion layer 112, the media layer 54 and the lower perfusion layer 116 are shown having oval through openings, the openings may optionally be round openings.

The cells are positioned within the well openings 82 and are attached to the top surface of the second membrane 90. The cell wells 74 around the well openings 82 provide a reservoir of liquid media to maintain the cells. The lower membrane 90 may be a hydrophilic PTFE membrane treated to allow the cells to better attach to it. The upper membrane 70 may also be a PTFE membrane, but without treatment. Both membranes are permeable to gases and liquid. The media wells 54 above the upper membrane 70 contain liquid media. The upper membrane 70 reduces or avoids shear stress on the cells in the well openings 82, as the liquid media diffuses through the membrane. It also reduces risks of displacing cells or exposing the cells to temperature shock via movement of gas and/or liquid into and out of the cell wells 74. The bottom layer 100 may be transparent to provide a bottom up site line to the cells in the well openings 82, to allow for optical detection of cell responses to a gas or liquid stimulant provided via the stimulation channel 96. The stimulation channel 96, air inlet holes 66, air outlet holes 68, the first perfusion channel 114 and the second perfusion channel 118, function in the same way as described above relative to FIG. 4. The layers in FIG. 16 may be adhered together or otherwise joined to form the multi-layer structure shown.

In the biochips shown in FIGS. 4 and 16, by having the inflow perfusion and the outflow perfusion on separate vertical levels it is possible to create a perfusion media flow that moves vertically (downwards or upwards). When the inflow and outflow perfusion are within the same layer as in FIG. 3A, newly introduced media may be the same media that is perfused outward without ever reaching the cells. Effective media exchange is impaired because the flow is laminar. Inward and outward perfusion may be performed at different times to avoid this result.

In FIG. 16, used media in the cell wells 74 flows up through the upper membrane 70 and can be removed (via aspiration or pumping) via second perfusion channel 118. Fresh media may be provided flowing down from the first perfusion channel 114, through the membrane 70 and into the cell wells 74. Media flow through the cell wells 74 is quasi turbulent, helping to flush out used media and replace it with fresh media, and with reduced mixing between the used media and the fresh media.

In use, after the biochip 50, 110 or 130 is assembled and ready for use, cells 30 are placed into the cell wells 74 or 150. The cells are seeded on top of the membrane 90 or 140, and the cells bind or attach to the membrane. A foil or pierceable seal layer may be adhered onto the top surface of the cover 52 to seal the wells, as well as the air or gas inlet and outlet holes 66 and 68, and the perfusion inlet and outlet holes 58 and 60. The foil or seal layer, if used, also prevents light from entering the top of biochip. This may reduce evaporation and help to avoid stray light affecting the signal from the photodetectors. The biochip is then effectively sealed against the environment.

The biochip is inserted into a VOC detection system, such as the system 20 shown in FIGS. 1 and 2. The system 20 moves to pierce the seal layer on the cover 52, if used, to connect perfusion inlet and outlet lines to the perfusion inlet and outlet holes 58 and 60. Optionally simultaneously, the system connects air sample inlet and outlet lines to the air inlet and outlet holes 66 and 68. An air sample is introduced into the stimulation channel 96. VOC's in the air sample (if present) diffuse through the membrane 90 and contact the cells 30 on top of the membrane. The cells 30 are genetically modified living cells expressing an odorant receptor capable of binding to the VOC. A fluorescent reporter fluoresces in response to binding the VOC to the odorant receptor. LEDs 40 project light from below through optical elements, as shown in FIG. 2. Optical sensors or photodiodes 48 below the biochip detect the fluorescent light which passes through the membrane 90, the stimulation channel 96 and the transparent bottom layer 100. The optical sensors are electrically connected to the system elements shown in FIGS. 1 and 2. The system detects and identifies the VOC by processing signals from the optical sensors.

The biochip may be manufactured as a disposable unit intended for replacement e.g., every 30 days. Although biochips described are designed for operation in the detection system 20 shown in FIGS. 1, 2, they may also be used in other systems as well. In some designs the foil or pierceable seal layer may itself be the cover 52, that is with the foil layer attached directly to the media layer 54.

As used here, layer means a component which may or may not have flat top and bottom surfaces, and which may or may not be discrete and separately identifiable apart from other components or sections of biochip. For example, the present biochips may be manufactured using rapid prototyping techniques, stereolithography, etc. which provide an integral end product without necessarily showing separate layers. The terms inlet and outlet are used here for purpose of description, without limitation as to direction of flow.

Thus, novel designs and methods have been shown and described. Various changes and substitutions may be made without departing from the spirit and scope of the invention. The invention, therefore, should not be limited, except to the following claims and their equivalents.

1. A biochip comprising:
a top layer;
a first perfusion layer between a the top layer and a media layer, the first perfusion layer having a first perfusion channel;
a second perfusion layer between the media layer and a first membrane, the second perfusion layer having a second perfusion channel;
a well layer between the first membrane and a second membrane, the well layer having a plurality of wells, each well containing genetically modified living cells expressing an odorant receptor capable of binding to a volatile organic compound, and a fluorescent reporter that fluoresces in response to binding of a volatile organic compound to the odorant receptor, the cells on the second membrane; and
a stimulation layer between the second membrane and a transparent layer,
the stimulation layer having a stimulation channel, each of the wells aligned over a portion of the stimulation channel.

2. The biochip of claim 1 wherein the first perfusion layer, the media layer, the second perfusion layer and the well layer, each have an air inlet hole and an air outlet hole, the air inlet and air outlet holes vertically aligned and leading into a stimulation channel inlet and a stimulation channel outlet, respectively.

3. The biochip of claim 2 wherein the stimulation channel includes a well region aligned under each of the wells, each well region having a diameter greater than the width of the stimulation channel.

4. The biochip of claim 3 wherein each of the first perfusion layer, the media layer, and the second perfusion layer has a through opening aligned over each of the wells.

5. The biochip of claim 4 further comprising a perfusion outlet hole in the first perfusion layer and in the media layer, the perfusion outlet hole aligned with the second perfusion channel.

6. The biochip of claim 1 wherein the well layer includes an upper well layer and a lower well layer, the wells formed in the upper well layer and the wells having a first diameter, and the lower well layer having well openings, the well openings having a second diameter less than the first diameter.

7. The biochip of claim 5 wherein the first perfusion channel extends from a first perfusion channel inlet to each of the through openings in the first perfusion channel layer, and the second perfusion channel extends from each of the through openings in the second perfusion channel layer to the perfusion outlet hole.

8. The biochip of claim 1 wherein the first membrane comprises a PTFE membrane and the second membrane comprises a PTFE membrane treated to promote cell adhesion.

9. The biochip of claim 5 wherein the first perfusion channel has a first perfusion channel inlet, and wherein the top layer seals off the air inlet hole, the air outlet hole, the first perfusion channel inlet and the perfusion outlet hole, sealing off the wells from the environment.

10. The biochip of claim 9 wherein areas of the top layer overlying the first perfusion channel inlet, the air inlet hole, the air outlet hole, and the perfusion outlet hole, are pierceable.

11. The biochip of claim 9 wherein the areas of the top layer overlying the first perfusion channel inlet, the air inlet hole, the air outlet hole, and the perfusion outlet hole, comprises a metal foil.

12. The biochip of claim 1 wherein each layer is adhered to one or more adjoining layer.
